(19) United States
(12) Patent Application Publication
(10) Pub. No.: US 2014/0186326 A1
(43) Pub. Date: Jul. 3, 2014

(54) MODIFIED ACID ALPHA GLUCOSIDASE WITH ACCELERATED PROCESSING

(71) Applicants: William Canfield, Oklahoma City, OK (US); Mariko Kudo, Oklahoma City, OK (US); Rodney Moreland, Bridgewater, NJ (US)

(72) Inventors: William Canfield, Oklahoma City, OK (US); Mariko Kudo, Oklahoma City, OK (US); Rodney Moreland, Bridgewater, NJ (US)

(21) Appl. No.: 14/113,360

(22) PCT Filed: Apr. 20, 2012
(86) PCT No.: PCT/US12/34479

§ 371 Date: Dec. 4, 2013

Related U.S. Application Data
(60) Provisional application No. 61/478,336, filed on Apr. 22, 2011.

(51) Int. Cl. C12N 9/26

(52) U.S. Cl. CPC C12N 9/2408
USPC 424/94.61; 536/23.2; 435/201; 435/369

(57) ABSTRACT

A modified human acid alpha-glucosidase polypeptide is provided, as well as methods of making and using modified human acid alpha-glucosidase to treat glycogen storage disorders.

This application claims the benefit of priority of U.S. Provisional Patent Application No. 61/478,336, filed Apr. 22, 2011, which is hereby incorporated by reference in its entirety.

This disclosure relates in general to modified human acid alpha-glucosidase and its use in treating glycogen storage diseases.

Pompe disease, also known as glycogen storage disease (GSD) type II and acid maltase deficiency, is an autosomal recessive metabolic myopathy caused by a deficiency of the lysosomal enzyme acid alpha-glucosidase (GAA). GAA is an exo-1,4 and 1,6-α-glucosidase that hydrolyzes glycogen to glucose in the lysosome. Deficiency of GAA leads to glycogen accumulation in lysosomes and causes progressive damage to respiratory, cardiac, and skeletal muscle. The disease ranges from a rapidly progressive infantile course that is usually fatal by 1-2 years of age to a more slowly progressive and heterogeneous course that causes significant morbidity and early mortality in children and adults. Hirschhorn R R, The Metabolic and Molecular Bases of Inherited Disease, 3: 3389-3420 (2001, McGraw-Hill); Van der Ploeg and Reuser, Lancet 372: 1342-1351 (2008).

The steps involved in the biosynthesis, targeting, and lysosomal processing of GAA are complex. The primary translation product of human GAA is a 952 amino acid polypeptide containing seven consensus N-glycosylation sites. Moreland et al., J. Biol. Chem. 280: 6780-6791 (2005). The N-glycans on GAA include complex and high-mannose type glycans, some of which are modified by mannose 6-phosphate. GAA is targeted to the lysosome via the cation-independent mannose 6-phosphate receptor. In the lysosome, the enzyme undergoes further processing by proteases and glycosidases, resulting in a mature peptide capable of increased glycogen clearance.

FIG. 1 shows a schematic of the GAA processing pathway. Moreland et al., 2005. Typically, GAA undergoes up to four cleavage events during processing. First, the primary GAA translation product is cleaved at around amino acid 57 to form a precursor with an apparent molecular weight of 100 to 110-kDa. Next, the 100 to 110-kDa precursor is cleaved around amino acids 113 and 122 to form a 3.9-kDa (aa 78-113) and a 95-kDa (aa 122-952) portion. The 95-kDa polypeptide may then be cleaved around amino acids 781 and 792 to yield 76-kDa (aa 122-781) and 19.4-kDa (aa 792-952) fragments. The 76-kDa species remains associated with the 19.4- and 3.9-kDa polypeptides. An additional proteolytic cleavage converts the 76-kDa to a 70-kDa (aa 204-781) species that remains associated with 19.4-, 10.4-, and 3.9-kDa polypeptides.

Current human therapy for treating Pompe disease involves administration of recombinant human GAA (e.g., MYOZYME™). Although recombinant human GAA effectively reduces glycogen accumulation in patients, it is not fully processed to the 70-kDa form upon administration. Because the affinity of GAA for glycogen may significantly increase as a result of protease processing (Moreland et al., 2005; Wisselaar et al., J. Biol. Chem. 268: 2223-2231 (1993)), increasing the rate of recombinant human GAA processing could allow for improved therapeutic efficacy of GAA, including lower doses and/or less frequent administration of GAA therapy.

Accordingly, we herein describe modified GAA polypeptides that are processed more rapidly than unmodified human GAA.

Certain embodiments include a human acid alpha-glucosidase or a catalytically-active fragment thereof having a modification at or near an N-terminal 70-kDa processing site. In some embodiments, a polypeptide is provided comprising a human acid alpha-glucosidase (GAA) or a catalytically-active fragment thereof having a modification at or near an N-terminal 70-kDa processing site. The catalytically-active fragment may be chosen from a 70-kDa, 76-kDa, 82-kDa, 95-kDa or any other catalytically-active fragment. In certain embodiments, the polypeptide further comprises a receptor targeting sequence. In some embodiments, the receptor targeting sequence is an IGF2 sequence.

In certain instances, the modification results in increased hydrophobicity at or near an N-terminal 70-kDa processing site. In some instances, the polypeptide is modified at one or more amino acids corresponding to positions 195-209 of SEQ ID NO: 1. In further embodiments, the modification is at one or more amino acids corresponding to amino acid positions 200-204 of SEQ ID NO: 1. In certain embodiments, the modification is at the amino acid corresponding to position 201 of SEQ ID NO: 1. In further embodiments, the modification is substitution of one or more amino acids with a more hydrophobic amino acid. In other embodiments, the modification is insertion of one or more hydrophobic amino acids. In even further embodiments, the hydrophobic amino acid is chosen from leucine and tyrosine.

In certain embodiments, the polypeptide has at least 80% identity to at least 500 amino acids of SEQ ID NO: 1. In some instances, the polypeptide has at least 90% identity to at least 500 amino acids of SEQ ID NO: 1. In other instances, the polypeptide has at least 95% identity to at least 500 amino acids of SEQ ID NO: 1.

In certain embodiments, the polypeptide exhibits more rapid lysosomal protease processing when compared to an unmodified human acid alpha-glucosidase. In some embodiments, at least 50% of the polypeptide is proteolytically processed to a 70-kDa form within 20 hours of administration. In other embodiments, substantially all the polypeptide is proteolytically processed to a 70-kDa form within 55 hours of administration.

Some embodiments include polypeptides conjugated to an oligosaccharide comprising at least one mannose-6-phosphate.

In certain embodiments, a nucleic acid is provided encoding a modified GAA polypeptide. In further embodiments, a host cell stably transfected with the nucleic acid is provided. In further embodiments, the host cell is capable of secreting modified GAA.

In certain embodiments, a method of reducing or preventing glycogen accumulation in a tissue is provided, comprising administering an effective amount of a polypeptide as described herein to a patient in need thereof. In further embodiments, the patient has a glycogen storage disease. In still further embodiments, the glycogen storage disease is Pompe disease.

In other embodiments, a method is provided for treating a glycogen storage disease, comprising administering a therapeutically effective amount of a modified GAA to a patient in need thereof. In further embodiments, the glycogen storage disease is Pompe disease. In other embodiments, a pharmaceutical composition is provided, comprising a modified GAA as described herein for use in treating a glycogen storage disease. In some embodiments, the polypeptide is lyophilized.

### BRIEF DESCRIPTION OF THE DRAWINGS

FIG. 1 is a diagram showing a model for the maturation of native human GAA.

FIG. 2 shows SDS-PAGE of recombinant GAA (lane 1), human placental GAA (lane 2), and bovine testes GAA (lane 3).

FIG. 3A shows an alignment of human GAA from amino acids 197 to 206 with GAAs from mouse, hamster, bovine, and quail. FIG. 3B shows the results of a western blot comparing different processed GAAs. Lane 1 shows human GAA purified from placenta. Lanes 2 and 3 are control GAAs purified from 293T cells transfected with wild-type human GAA constructs. Lanes 4-7 are modified GAAs purified from 293T cells transfected with human GAA constructs where the histidine at amino acid 201 was changed to the following amino acids: arginine (lane 4), leucine (lane 5), tyrosine (lane 6), and lysine (lane 7).

FIG. 4 shows the biosynthesis of rhGAA(H201L) and rhGAA (WT) in stably transfected CHO cells.

FIG. 5 shows Pompe fibroblast uptake and processing of rhGAA (WT) and rhGAA (H201L).

FIG. 6 shows the results of Western blots probed with anti-GAA 183-200 (FIG. 6A) and monoclonal antibody GAA1 (FIG. 6B).

FIG. 7 is a schematic of a processing model for rhGAA(H201L).

### DESCRIPTION OF THE EMBODIMENTS

To assist in understanding the present disclosure, certain terms are first defined. Additional definitions are provided throughout the application.

As used herein, the term “N-terminal 70-kDa processing site” refers to the recognition site for the proteolytic enzyme(s) that cleave GAA at the position corresponding to amino acids 200 to 204 of SEQ ID NO: 1 (native human GAA).

As used herein, the term “modified GAA” refers to human GAA and GAA variants having at least one amino acid at or near the N-terminal 70-kDa processing site that differs from the amino acid found in native human GAA. Modified GAA is also referred to as “modified human GAA” in the description. The term “modified GAA” includes full-length GAA polypeptides that contain signal sequences, as well as partially-processed GAA polypeptides as secreted from cells.

As used herein, the singular forms “a,” “an,” and “the” include plural referents unless the content clearly dictates otherwise. Thus, for example, reference to a method containing “a compound” includes a mixture of two or more compounds. The term “or” is generally employed in its sense including “and/or” unless the content clearly dictates otherwise.

Throughout the specification, protein and polypeptide sizes are provided in “kDa” units. One of skill in the art will recognize that these sizes are based on apparent molecular weight of polypeptides in electrophoresis assays such as SDS-PAGE (see, e.g., Moreland et al., 2005). Exact molecular weights will depend on glycosylation state and other parameters such as association with other polypeptides, and can be determined by various methods that are well-known to those of skill in the art.

All references cited herein are incorporated by reference in their entirety. To the extent publications and patents or patent applications incorporated by reference contradict the invention contained in the specification, the specification will supersede any contradictory material.

### I. ACID ALPHA-GLUCOSIDASE (GAA)

As described above, GAA is a lysosomal enzyme involved in clearance of glycogen. The term GAA encompasses both full-length, wild-type forms of the protein, as well as other catalytically-active variants. Catalytically-active GAA and GAA variants will at least retain catalytic activity toward glycogen. Numerous variants of native human GAA are known to those of skill in the art, including those that have been truncated, fused or conjugated to other polypeptides, altered in their amino acid sequences, or altered recombinantly or chemically. For instance, it is known that at least 77 N-terminal amino acids can be removed from native human GAA (SEQ ID NO: 1) without losing activity. Moreland et al., 2005. In addition, conjugates and fusion proteins have been described. In some embodiments, a GAA or catalytically-active fragment of GAA can be conjugated or fused to a receptor targeting sequence. In some instances, the receptor targeting sequence can be recognized by a cellular receptor. For example, a truncated GAA may be fused to an IGF2 domain as described in U.S. Pat. No. 7,785,856, which is incorporated by reference in its entirety. GAA has also been altered to add synthetic moieties, carbohydrate moieties and/or increased levels of mannose-6-phosphate. For example, lysosomal enzymes with modified carbohydrate moieties containing increased levels of mannose-6-phosphate are described in U.S. Pat. Nos. 7,001,994; 7,723,296; 7,786,277; U.S. Patent Publication 2010/0173385; and PCT Publication 2010/075010, which are incorporated by reference in their entirety.

In certain embodiments, the GAAs described herein have at least 80%, 90%, 95%, or 99% identity to a human GAA or GAA variant. In some instances, the GAA has at least 80%, 90%, 95%, or 99% identity to at least 500, 550, 600, 650, 700, 750, 800, 850, or 900 amino acids of SEQ ID NO: 1.

Any of the catalytically-active human GAAs described in this section can be used as the base sequence for a modified GAA described herein. One of skill in the art will recognize which GAA variants are suitable for use in the invention. Where a base GAA sequence has a different length or glycosylation pattern compared to native human GAA, the processed polypeptides will have sizes that vary accordingly.

### II. MODIFIED GAA

In various embodiments, a polypeptide comprising a modified human GAA is provided that is modified at or near the N-terminal 70-kDa processing site. The region “near” the N-terminal 70-kDa processing site includes up to 5 amino acids upstream or downstream of the N-terminal 70-kDa processing site. In certain embodiments, the region at or near the N-terminal 70-kDa processing site includes the amino acids corresponding to positions 195-209 of SEQ ID NO: 1.

The modified GAAs described herein are processed more rapidly than unmodified GAA. In certain embodiments, the modified GAA has increased hydrophobicity at or near the N-terminal 70-kDa processing site. In some embodiments, the modified GAA has a faster rate of proteolytic processing to a 70-kDa mature form. In some embodiments, and depending on the starting sequence, the modified GAA is processed to a variant of the 70-kDa mature form. The modified GAA may be processed such that the mature polypeptide remains associated with additional polypeptide fragments. In certain embodiments, the modified GAA is processed via the same pathway as unmodified GAA. In other embodiments, the modified GAA is processed via different intermediates compared to unmodified GAA. For example, a modified full-length GAA may be processed via 76-kDa or 82-kDa intermediates, or both. The modified GAA may be recognized by the same proteases as unmodified GAA, and processed in the same or a different order.

In certain embodiments, GAA is modified to increase its hydrophobicity at or near the N-terminal 70-kDa processing site by substituting at least one amino acid with a more hydrophobic amino acid. In some embodiments, the substitution may be made within 5 amino acids upstream or downstream of the N-terminal 70-kDa processing site. In certain examples, the amino acid substitution may be made at an amino acid corresponding to position 195 to 209 of SEQ ID NO: 1. In other instances, the amino acid substitution may be made at an amino acid corresponding to position 200 to 204 of SEQ ID NO: 1. In further embodiments, the modified human GAA contains a hydrophobic amino acid at the position corresponding to amino acid position 201 of SEQ ID NO: 1. In some embodiments, GAA is modified by inserting one or more hydrophobic amino acids at or near the N-terminal 70-kDa processing site. Additional modifications include deletion of one or more amino acids at or near the N-terminal 70-kDa processing site.

In certain embodiments, a modified human GAA is provided containing a hydrophobic amino acid (natural or synthetic) at more than one position at the N-terminal 70-kDa processing site, or within 5 amino acids of the N-terminal 70-kDa processing site. In one embodiment, one of the modified amino acids is at the position corresponding to amino acid 201 of SEQ ID NO: 1.

In various embodiments the hydrophobic amino acid is chosen from valine, leucine, isoleucine, methionine, phenylalanine, tryptophan, tyrosine, cysteine or alanine. In further embodiments, the hydrophobic amino acid is leucine or tyrosine. In some embodiments, the modified human GAA contains a synthetic or non-natural amino acid that exhibits hydrophobic properties. Generally, the substituted amino acid is more hydrophobic than the wild-type amino acid, and thus increases the hydrophobicity at or near the N-terminal 70kDa processing site.

In one exemplary embodiment, the modified GAA has a leucine at the position corresponding to amino acid 201 of SEQ ID NO: 1. In another embodiment, the modified GAA has a tyrosine at the position corresponding to amino acid 201 of SEQ ID NO: 1.

In certain embodiments, modified human GAAs are provided having at least 80%, 90%, 95%, or 99% homology to at least 500, 550, 600, 650, 700, 750, 800, 850, or 900 amino acids of SEQ ID NO: 1, and wherein the modified human GAA has at least one amino acid in the N-terminal 70-kDa processing site substituted with a more hydrophobic amino acid.

In some embodiments, at least 50% of the modified human GAA is processed to a 70-kDa form in the lysosome within 20, 30, or 40 hours. In still further embodiments, substantially all of the modified human GAA is processed to a 70-kDa form in the lysosome within 55, 65, or 75 hours.

In certain embodiments, a modified human GAA of the invention can be identified by its more rapid proteolytic processing to a mature 70-kDa form, or a corresponding variant thereof. In other embodiments, a modified human GAA as described herein can be identified by the production of an 82-kDa intermediate polypeptide that is not produced during proteolytic processing of native human GAA. In further embodiments, a modified human GAA can be identified by the absence of a 76-kDa intermediate polypeptide that is produced during proteolytic processing of unmodified human GAA.

### III. PRODUCTION OF MODIFIED GAA

In various embodiments, a modified GAA polypeptide can be produced according to methods known to one of skill in the art. For example, a modified GAA polypeptide can be expressed and secreted from cell lines stably transfected with nucleic acids encoding modified GAA. Suitable cell lines include fibroblast cells, Chinese Hamster Ovary (CHO) cells, 293T cells, or plant cells, among others recognized by those of skill in the art. Exemplary cell lines and production methods are described in U.S. Pat. Nos. 7,351,410 and 7,138,262; and U.S. Patent Publication No. 2010/0196345, which are hereby incorporated by reference in their entirety. In certain embodiments, a nucleic acid encoding a modified GAA is inserted in a plasmid or vector containing the appropriate promoters and regulatory sequences for expression from a cell line. Promoters useful for producing modified GAA in mammalian cell lines include the rpS21 and beta-actin promoters (see, e.g., U.S. Pat. No. 7,423,135), among many others recognized by those of skill in the art. In certain embodiments, modified GAA is further altered to increase or decrease levels of glycosylation or mannose 6-phosphate, thereby enhancing secretion and/or lysosomal targeting.

### IV. PHARMACEUTICAL COMPOSITIONS

In certain embodiments, the modified GAA is present in a pharmaceutical composition comprising at least one additive such as a filler, bulking agent, disintegrant, buffer, stabilizer, or excipient. Standard pharmaceutical formulation techniques are well known to persons skilled in the art (see, e.g., 2005 Physicians' Desk Reference®, Thomson Healthcare: Montvale, N.J., 2004; Remington: The Science and Practice of Pharmacy, 20th ed., Gennado et al., Eds. Lippincott Williams & Wilkins: Philadelphia, Pa., 2000). Suitable pharmaceutical additives include, e.g., mannitol, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In certain embodiments, the pharmaceutical compositions may also contain pH buffering reagents and wetting or emulsifying agents. In further embodiments, the compositions may contain preservatives or stabilizers.

In some embodiments, pharmaceutical compositions comprising modified human GAA may further comprise one or more of the following: mannitol, polysorbate 80, sodium phosphate dibasic heptahydrate, and sodium phosphate monobasic monohydrate. In another embodiment, pharmaceutical compositions may contain 10 mM Histidine pH 6.5 with up to 2% glycine, up to 2% mannitol, and up to 0.01% polysorbate 80. Additional exemplary pharmaceutical compositions can be found in PCT Publication No. 2010/075010.

The formulation of pharmaceutical compositions may vary depending on the intended route of administrations and other parameters (see, e.g., Rowe et al., Handbook of Pharmaceutical Excipients, 4th ed., APhA Publications, 2003.) In some embodiments, the modified GAA composition may be a lyophilized cake or powder. The lyophilized composition may be reconstituted for administration by intravenous injection, for example with Sterile Water for Injection, USP. In other embodiments, the composition may be a sterile, non-pyrogenic solution.

The pharmaceutical compositions described herein may comprise modified GAA as the sole active compound or may be delivered in combination with another compound, composition, or biological material. For example, the pharmaceutical composition may also comprise one or more small molecules useful for the treatment of Pompe disease and/or a side effect associated with Pompe disease or its treatment. In some embodiments, the composition may comprise miglustat and/or one or more compounds described in, e.g., U.S. Patent Application Publication Nos. 2003/0050299, 2003/0153768; 2005/0222244; or 2005/0267094. In some embodiments, the pharmaceutical composition may also comprise one or more immunosuppressants, mTOR inhibitors or autophagy inhibitors. Examples of immunosuppressants include rapamycin and velcade. Rapamycin is also an mTOR inhibitor.

### V. THERAPEUTIC METHODS

In some embodiments, a modified human GAA is used to reduce or prevent glycogen accumulation in a tissue of a patient. In other embodiments, modified human GAA is used to treat a glycogen storage disease. In further embodiments, the glycogen storage disease is Pompe disease. In exemplary embodiments, the modified GAA is subsequently processed into mature GAA in the lysosome after administration to the patient.

The modified GAA described herein may be administered by any suitable delivery system and may include, without limitation, parenteral (including subcutaneous, intravenous, intracranial, intramedullary, intraarticular, intramuscular, intrathecal, or intraperitoneal injection), transdermal, or oral (for example, in capsules, suspensions, or tablets). In one embodiment, the modified GAA is delivered by intravenous administration.

In additional embodiments, a nucleic acid encoding a modified GAA can be delivered to the patient. The nucleic acid may be delivered using a vector suitable for gene therapy. Examples of gene therapy methods are described in, e.g., U.S. Pat. Nos. 5,952,516; 6,066,626; 6,071,890; and 6,287,857.

Administration to a patient may occur in a single dose or in repeat administrations, and in any of a variety of physiologically acceptable salt forms, and/or with an acceptable pharmaceutical carrier and/or additive as part of a pharmaceutical composition.

The modified GAA compositions described herein are administered in therapeutically effective amounts. Generally, a therapeutically effective amount may vary with the subject's age, general condition, and gender, as well as the severity of the medical condition in the subject. The dosage may be determined by a physician and adjusted, as necessary, to suit observed effects of the treatment.

The modified GAAs described herein may be administered by intravenous infusion in an outpatient setting every, e.g., 1, 2, 3, 4, 5, 6, 7, 8, 9, 10, or more days, or by, e.g., weekly, biweekly, monthly, or bimonthly administration. The appropriate therapeutically effective dose of a compound is selected by a treating clinician and may range from approximately 1 μg/kg to approximately 500 mg/kg, from approximately 10 mg/kg to approximately 100 mg/kg, from approximately 20 mg/kg to approximately 100 mg/kg and from approximately 20 mg/kg to approximately 50 mg/kg. In some embodiments, the appropriate therapeutic dose is chosen from, e.g., 0.1, 0.25, 0.5, 0.75, 1, 5, 10, 15, 20, 30, 40, 50, 60, 70, and 100 mg/kg. Additionally, examples of specific dosages may be found in the Physicians' Desk Reference®.

In some embodiments, the methods comprise administering modified human GAA, thereby increasing the glycogen clearance in the subject by, e.g., at least 10%, 15%, 20%, 25%, 30%, 35%, 40%, 50%, 60%, 70%, 80%, 90%, or 100%, relative to endogenous activity. In some embodiments, the methods comprise administering modified human GAA, thereby increasing glycogen clearance in the subject by, e.g., at least 2, 3, 4, 5, 6, 7, 8, 9, 10, 20, 30, 40, 50, 100, or 1000 fold, relative to endogenous activity. The increased glycogen clearance may be determined by, e.g., a reduction in clinical symptoms or by an appropriate clinical or biological assay such as a lysosome glycogen storage assay.

In certain embodiments, increased glycogen clearance after treatment of a patient with a pharmaceutical composition comprising modified human GAA may be determined by biochemical (see, e.g., Zhu et al., J. Biol. Chem. 279: 50336-50341 (2004)) or histological observation of reduced lysosomal glycogen accumulation in, e.g., cardiac myocytes, skeletal myocytes, or skin fibroblasts. GAA activity may also be assayed in, e.g., a muscle biopsy sample, in cultured skin fibroblasts, in lymphocytes, and in dried blood spots. Dried blood spot assays are described in e.g., Umpathysivam et al., Clin. Chem. 47:1378-1383 (2001) and Li et al., Clin. Chem. 50:1785-1796 (2004). Treatment of Pompe disease may also be assessed by, e.g., serum levels of creatinine kinase, gains in motor function (e.g., as assessed by the Alberta Infant Motor Scale), changes in left ventricular mass index as measured by echocardiogram, and cardiac electrical activity, as measured by electrocardiogram. Administration of a pharmaceutical composition comprising modified human GAA may also result in a reduction in one or more symptoms of Pompe disease such as cardiomegaly, cardiomyopathy, daytime somnolescence, exertional dyspnea, failure to thrive, feeding difficulties, “floppiness,” gait abnormalities, headaches, hypotonia, organomegaly (e.g., enlargement of heart, tongue, liver), lordosis, loss of balance, lower back pain, morning headaches, muscle weakness, respiratory insufficiency, scapular winging, scoliosis, reduced deep tendon reflexes, sleep apnea, susceptibility to respiratory infections, and vomiting.

In certain embodiments, the methods comprise administering pharmaceutical compositions comprising modified human GAA with one or more additional therapies. The one or more additional therapies may be administered concurrently with (including concurrent administration as a combined formulation), before, or after the administration of the modified human GAA. In some instances, an additional therapy can be administered between doses of modified GAA. For example, small molecule therapy may be used to slow reaccumulation of glycogen, allowing for less frequent doses of the modified GAA.

In some embodiments, the methods comprise treating a subject with an antipyretic, antihistamine, and/or immunosuppressant (before, after, or during treatment with a modified human GAA described supra). In certain embodiments, a subject may be treated with an antipyretic, antihistamine, and/or immunosuppressant prior to treatment with a modified human GAA in order to decrease or prevent infusion associated reactions. For example, subjects may be pretreated with one or more of acetaminophen, azathioprine, cyclophosphamide, cyclosporin A, diphenhydramine, methotrexate, mycophenolate mofetil, oral steroids, or rapamycin.

In some embodiments, the methods comprise treating a subject (before, after, or during treatment with a modified human GAA) with small molecule therapy and/or gene therapy, including small molecule therapy and gene therapy directed toward treatment of a glycogen storage disorder. Small molecule therapy may comprise administration of miglustat and/or one or more compounds described in, e.g., U.S. Patent Application Pub. Nos. 2003/0050299, 2003/0153768; 2005/0222244; and 2005/0267094. Gene therapy may be performed as described in, e.g., U.S. Pat. Nos. 5,952,516; 6,066,626; 6,071,890; and 6,287,857; and U.S. Patent Application Pub. No. 2003/0087868.

### VI. EXAMPLES

The following examples serve to illustrate, and in no way limit, the present disclosure.

### Example 1

### Materials and Methods

A. Assay Reagents and Materials

Concanavalin A, DEAE-Sepharose FF, and Superdex 200 prep grade were obtained from Amersham Pharmacia Biotech (Piscataway, N.J.). α-methylglucoside, benzamidine, and 4-methylumbelliferyl α-D-glucoside were obtained from Sigma-Aldrich (Saint Louis, Mo.). Other chemicals were reagent grade or better and were from standard suppliers. SDS-PAGE gels were obtained from Invitrogen (San Diego, Calif.). Roller bottles were obtained from Corning (Corning, N.Y.). Dulbecco's Modified Eagle's Medium (DMEM) and fetal bovine serum (FBS) were obtained from JRH Biosciences (Lenexa, Kans.). Pompe fibroblasts (GM00248) were obtained from Coriell Cell Repositories (Camden, N.J.).

B. Acid α-glucosidase Activity and Protein Assay

Acid α-glucosidase was assayed fluorimetrically in a microtiter plate using 4-methylumbelliferyl α-D-glucoside as previously described. Oude Elferink et al., Eur. J. Biochem. 139: 489-495 (1984). Protein concentration was estimated by absorbance at 280 nm assuming E^{1%}=10 or using the Micro-BCA assay standardized with bovine serum albumin. Smith et al., Anal. Biochem. 150: 76-85 (1985).

C. SDS-Polyacrylamide Gel Electrophoresis

Reduced and non-reduced samples and molecular weight markers (Amersham Pharmacia Biotech) were applied to a 4-20% or 10% Tris-Glycine SDS-PAGE gel. Electrophoresis was performed at 150 volts for 1.5 hours and proteins were visualized with either Coomassie blue or silver stain. Blum et al., Electrophoresis 93-99 (1987).

D. Isolation of Recombinant and Placental GAA

The production and purification of recombinant and human placental GAA was as previously described. Martiniuk et al., Archives of Biochem. and Biophys. 231: 454-460 (1984); Mutsaers et al., Biochimica et Biophysica Acta 911: 244-251 (1987); Moreland et al., (2005).

E. Antibodies and Western Blot Analysis

As previously described (Moreland et al., 2005), rabbits were immunized with synthesized peptides coupled to KLH. The sequence for each peptide was as follows: anti-GAA 57-74 (QQGASRPGPRDAQAHPGR (SEQ ID NO: 2)), anti-GAA 78-94 (VPTQCDVPPNSRFDCA (SEQ ID NO: 3)), and anti-GAA 183-200 (IKDPANRRYEVPLETPRV (SEQ ID NO: 4)). Goat polyclonal antibody was generated against purified human placental GAA. Monoclonal antibody GAA1 was previously described. Moreland et al., 2005. Western blots were performed as previously described. Moreland et al., 2005.

F. Fibroblast Uptake of rhGAA

For each time point, approximately 5×10⁵ Pompe fibroblasts in DMEM plus 10% FBS were incubated with 250 nM rhGAA(WT) or rhGAA(H201L). At 16 hours, the cells were washed and fresh media that did not contain GAA was added. At the designated time points, the cells were removed and washed 5 times with phosphate buffered saline and stored at −80° C. After the final time point, all cell pellets were thawed and lysed simultaneously with 0.25% Triton. Cellular debris was pelleted and western blot analysis was performed on supernatants from each time point with anti-GAA antibodies.

G. Preparation of Expression Constructs and Transient Transfections

Expression plasmids for recombinant GAA with and without amino acid substitution or deletions were made in pcDNA6 (Invitrogen), using standard procedures. Human kidney 293T cells were cultured in DMEM, supplemented with 10% FBS under 5% CO₂ at 37° C. Six micrograms of each plasmid were mixed with Fugene 6 transfection reagent (Roche) and added to 2.5×10⁶ cells in a 10 cm dish. After 72 h the adherent cells were washed with PBS two times and lysed with PBS containing 0.25% Triton. The cellular debris was precipitated by centrifugation and the supernatants were stored at −20° C.

H. Metabolic Labeling and Immunoprecipitation

Stable CHO cell lines expressing rhGAA(WT) or rhGAA(H201L) were created following the method described previously. Qiu et al., J. Biol. Chem. 278: 32744-32752 (2003). Approximately 5×10⁶ cells in a 10 cm dish were incubated in DMEM lacking methionine and cysteine for 30 min. The cells were pulse-labeled for 2 h with 150 μCi/ml (1175 Ci/mmol Tran³⁵S Label) in DMEM deficient in methionine and cysteine. After the cells were washed with DMEM two times and the 0 h time point was taken, the cells were then incubated in DMEM without label at 37° C. At each time point, the cells were washed two times with PBS. The dishes were stored at −20° C. After the final time point, the cells were lysed with PBS containing 0.25% Triton (PBST). The cellular debris was removed by centrifugation and 60 μl of a 50% slurry of Concanavalin A Sepharose was added to the supernatant. After 2 hr incubation, the beads were washed 3 times with PBST. The labeled GAA was eluted with PBS containing 0.5 M α-methylglucoside. The GAA present in the eluent was then immunoprecipitated with affinity purified goat anti-GAA coupled to NHS-Sepharose. The immunoprecipitate was washed 3 times with PBST and 40 μl of 2×SDS sample buffer containing β-mercaptoethanol was added to the beads. The samples were boiled prior to western blot analysis.

I. Abbreviations

As used herein, “rhGAA” means recombinant human acid α-glucosidase. “CHO” means Chinese hamster ovary. “MSX” means methionine sulfoximine. “ERT” means enzyme replacement therapy.

As used herein, “GAA (H201R)” means a modified GAA having an amino acid substitution at position 201 from histidine to arginine. “GAA (H201L)” means a modified GAA having an amino acid substitution at position 201 from histidine to leucine. “GAA (H201Y)” means a modified GAA having an amino acid substitution at position 201 from histidine to tyrosine. “GAA (H201K)” means a modified GAA having an amino acid substitution at position 201 from histidine to lysine.

### Example 2

### Comparison of Human, Bovine and Hamster GAA

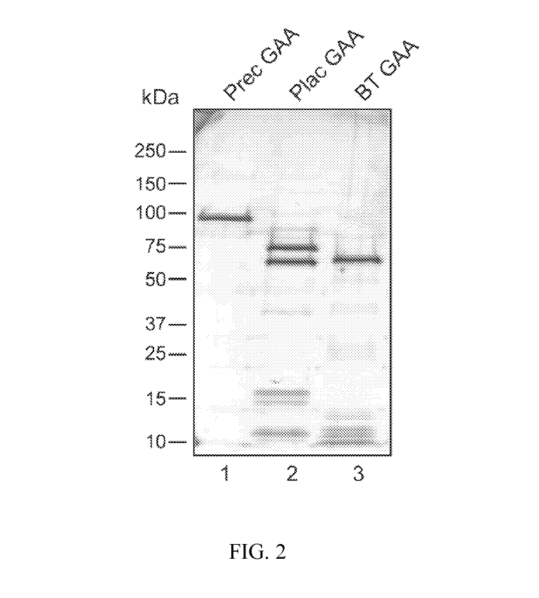

When GAA purified from placenta was examined by SDS-PAGE, two bands corresponding to polypeptides of 76- and 70-kDa were approximately in equal abundance (FIG. 2). Likewise, rhGAA over-expressed in Chinese hamster ovary (CHO) cells and purified from cell lysates has previously demonstrated 76- and 70-kDa bands. Moreland et al., 2005. By contrast, hamster GAA purified from CHO cells exists exclusively as the 70-kDa polypeptide. To determine if the predominance of the 70-kDa form was unique to hamster, bovine testis GAA was purified and characterized by reduced silver stained SDS-PAGE (4-20% acrylamide), and also shown to contain only the 70-kDa polypeptide (FIG. 2).

### Example 3

### The Amino Acid at Position 201 of GAA Affects the Efficiency of Conversion from the 76- to 70-kDa Form and Determines the Order of Proteolytic Cleavages

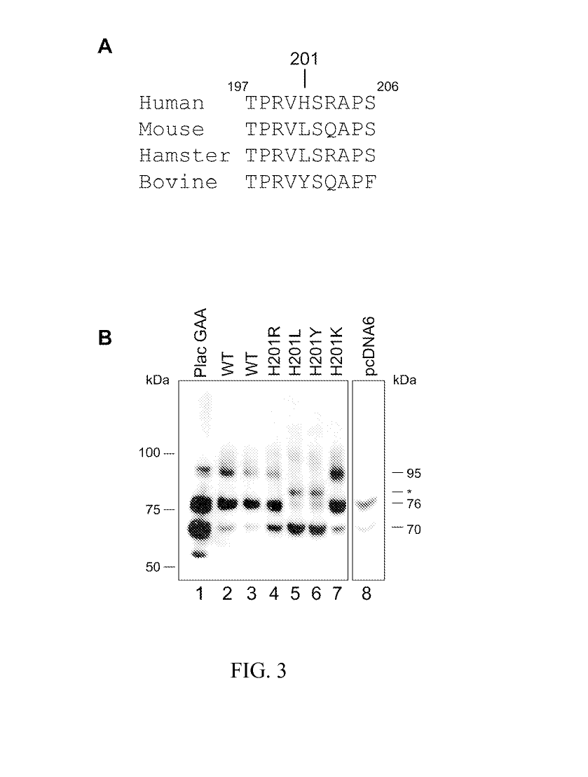

Alignment of mammalian GAA sequences at the proteolytic site between amino acids 197 and 206 (FIG. 3A) demonstrates that the retained sequences are highly conserved but that the excised sequences exhibit some variation. Human GAA contains a histidine at position 201 while hamster and bovine GAA have the hydrophobic residues leucine and tyrosine, respectively. To determine if these amino acid substitutions are responsible for species-specific differences in processing, GAA expression plasmids were constructed in which amino acid 201 was varied. The histidine was substituted with leucine (H201L), tyrosine (H201Y), arginine (H201R) or lysine (H201K). Human embryonic kidney cells (293T) were transfected with each construct followed by western blot analysis with a monoclonal antibody to GAA. Western blot analysis of the cell lysates indicated that when amino acid 201 in GAA was substituted with leucine or tyrosine, conversion from the 76- to 70-kDa form was dramatically more efficient compared to wild type (FIG. 3B, lanes 2-7). The hydrophobic amino acid substitution appeared to cause the formation of a new ˜82-kDa intermediate, as indicated by the asterisk (FIG. 3B). In the vector only control (FIG. 3, lane 8), nine times more cell lysate was loaded to visualize the endogenous GAA compared to the lysates from the transiently transfected 293T cells.

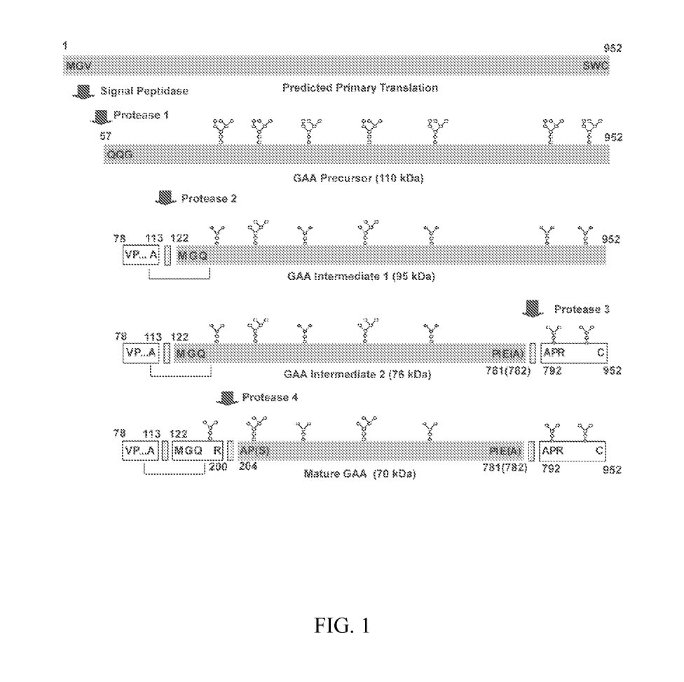

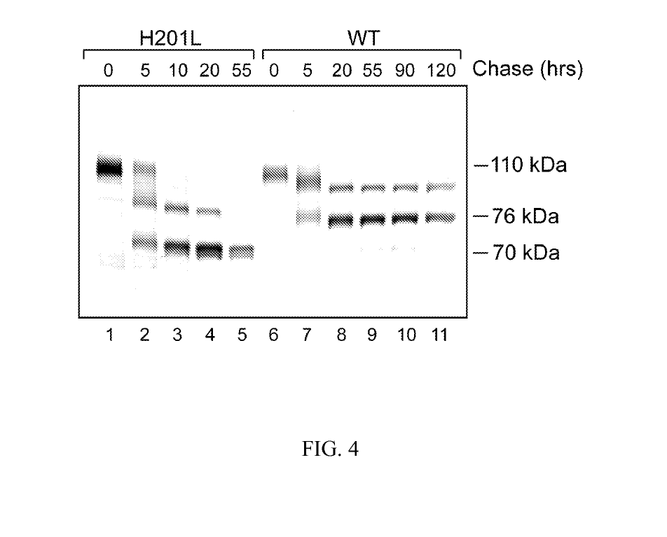

To characterize the rate of processing of wild type GAA compared to GAA(H201L), a pulse chase experiment was performed. Stable CHO cell lines expressing each GAA were radio-labeled for 2 hours with Tran³⁵S and chased for the times indicated with media that did not contain label (FIG. 4). rhGAA was purified from cell lysates by Con A followed by immunoprecipitation as described in Example 1. Time 0 hr was after the 2 hr pulse. At the 55 hour time point, GAA(H201L) was completely processed to the 70-kDa form, while very little of the wild type GAA was processed to the 70-kDa form after 120 hours. A 95-kDa species was observed in cells expressing the wild type GAA but was absent in the H201L. The identity of the 95-kDa intermediate has been previously characterized (Moreland et al., 2005) and is depicted in FIG. 1.

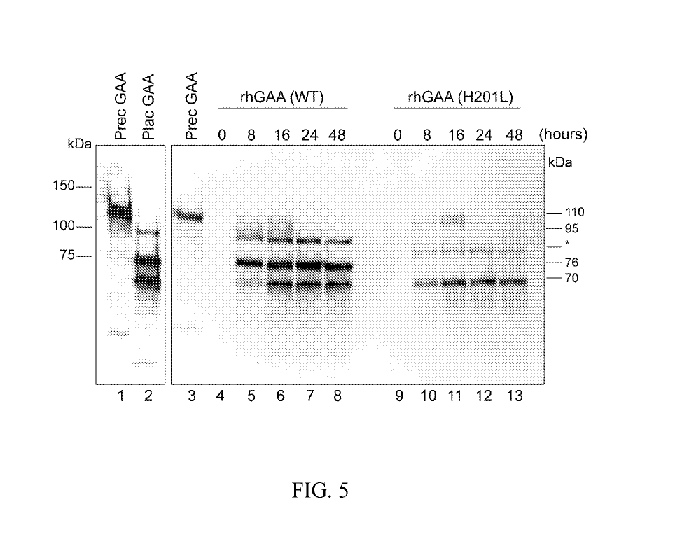

To determine if rhGAA(H201L) undergoes accelerated processing in uptake studies, the secreted form of rhGAA(H201L) and rhGAA(WT) were purified from stable recombinant CHO cell lines. Uptake studies were performed in Pompe fibroblasts because they are deficient in GAA (FIG. 5). As described in Example 1, GAA deficient Pompe fibroblasts (GM00248) were incubated with 250 nM rhGAA (WT) and rhGAA (H201L). At the designated time points the fibroblasts were collected and frozen at −80° C. A reduced SDS-PAGE (7.5% acrylamide) western blot of the cell lysates was probed with a monoclonal antibody to human GAA (the unknown epitope of which lies within amino acids 204-782). After uptake of GAA, the 95-kDa and 76-kDa forms were prominent for rhGAA(WT) and not observed for rhGAA(H201L) (FIG. 5, lanes 5-13). The difference in processing was again accompanied by the appearance of an ˜82-kDa intermediate with GAA(H201L).

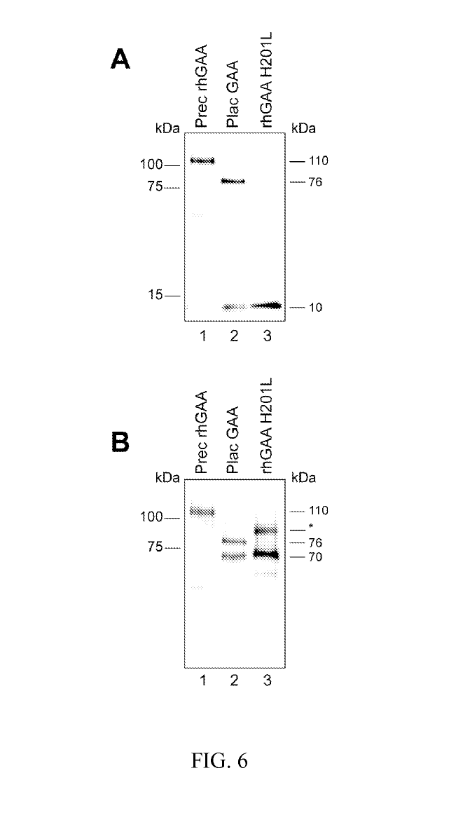

To characterize the ˜82-kDa intermediate, a western blot containing purified rhGAA, placental GAA, and rhGAA(H201L) was probed with an anti-GAA antibody that recognizes amino acids 183-200 (and thus binds the 10.4-kDa fragment released from fully processed GAA) (FIG. 6A). rhGAA, placental GAA, and mature rhGAA(H201L) were purified as described in Example 1. The 76-kDa species from placenta still contained amino acids 183-200, as indicated by antibody binding. In contrast, the 82-kDa intermediate did not contain these amino acids, as indicated by the lack of antibody binding. This is because cleavage of the antibody recognition site had already taken place, as evidenced by the ˜10-kDa band in lane 3. A separate monoclonal antibody to GAA demonstrated the presence of the 82-kDa intermediate in the sample of rhGAA(H201L) (FIG. 6B).

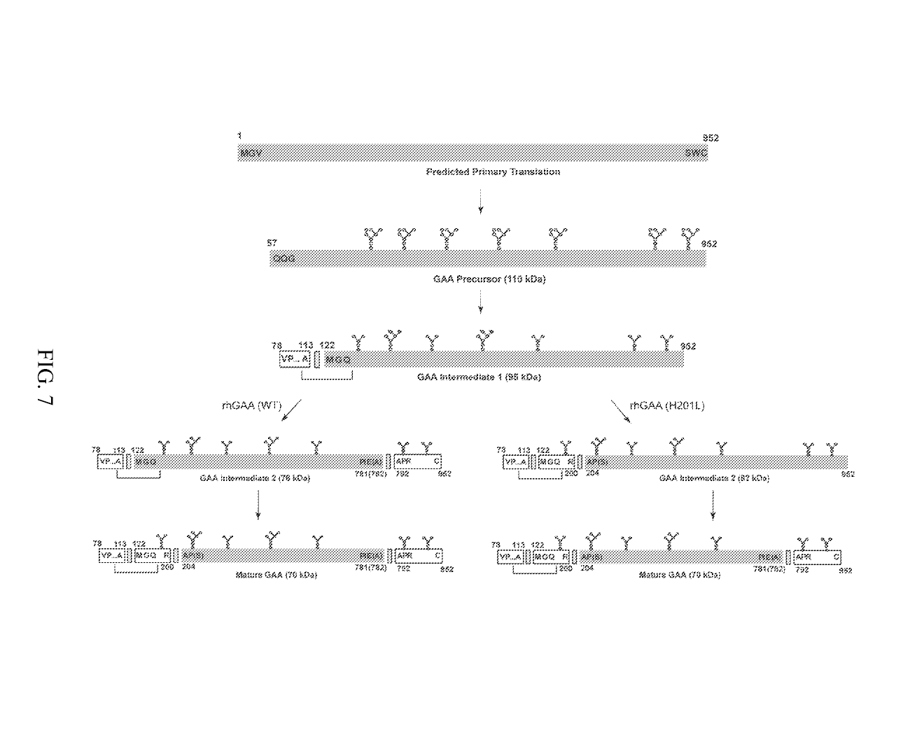

It can be concluded that the 82-kDa intermediate results from accelerated proteolysis at the cleavage site between amino acids 200 to 204. The cleavage takes place before the cleavage between amino acids 782 to 792. As shown in FIG. 6A, the 82-kDa polypeptide does not contain the ˜10-kDa fragment from amino acids 122-200. These results suggest an alternative processing pathway for rhGAA(H201L) as shown in FIG. 7. Processing of wild type vs. GAA(H201L) diverge after the 95-kDa intermediate. The wild type GAA is cleaved near the carboxyl terminus (between amino acids 781-792) to give the 76-kDa intermediate while GAA(H201L) is cleaved between amino acids 200-204 to give the 82-kDa intermediate. Both pathways ultimately result in mature 70-kDa GAA.

Other embodiments of the present disclosure will be apparent to those skilled in the art from consideration of the specification and practice of the embodiments disclosed herein. It is intended that the specification and examples be considered as exemplary only.

1. (canceled)

2. A polypeptide comprising a human acid alpha-glucosidase or a catalytically-active fragment thereof having a modification at or near an N-terminal 70-kDa processing site.

3. The polypeptide of claim 2, wherein the modification is increased hydrophobicity at or near the N-terminal 70-kDa processing site.

4. The polypeptide of claim 2, wherein the modification is at one or more amino acids corresponding to positions 195-209 of SEQ ID NO: 1.

5. The polypeptide of claim 4, wherein the modification is at one or more amino acids corresponding to positions 200-204 of SEQ ID NO: 1.

6. The polypeptide of claim 5, wherein the modification is at the amino acid corresponding to position 201 of SEQ ID NO: 1.

7. The polypeptide of claim 2, wherein the modification comprises
a) substitution of one or more amino acids with a more hydrophobic amino acid, or
b) insertion of one or more hydrophobic amino acids.

8. The polypeptide of claim 2, wherein the fragment is chosen from a 70-kDa, 76-kDa, 82-kDa, 95-kDa, or any other catalytically-active fragment of human acid alpha-glucosidase.

9. The polypeptide of claim 8, wherein the polypeptide further comprises a receptor targeting sequence.

10. The polypeptide of claim 9, wherein the receptor targeting sequence is IGF2.

11. The polypeptide of claim 2, wherein the polypeptide has at least 80% identity to at least 500 amino acids of SEQ ID NO: 1.

12-13. (canceled)

14. The polypeptide of claim 2, wherein the modified polypeptide exhibits more rapid lysosomal protease processing when compared to an unmodified human acid alpha-glucosidase.

15-16. (canceled)

17. The polypeptide of claim 2, wherein the polypeptide is conjugated to an oligosaccharide comprising at least one mannose-6-phosphate.

18. A nucleic acid encoding a polypeptide of claim 2.

19. A host cell stably transfected with the nucleic acid of claim 18.

20. (canceled)

21. A method of reducing or preventing glycogen accumulation in a tissue, comprising administering an effective amount of a polypeptide of claim 2 to a patient in need thereof.

22. The method of claim 21, wherein the patient has a glycogen storage disease.

23. The method of claim 22, wherein the glycogen storage disease is Pompe disease.

24. A method of treating a glycogen storage disease, comprising administering a therapeutically effective amount of a polypeptide of claim 2 to a patient in need thereof.

25. The method of claim 24, wherein the glycogen storage disease is Pompe disease.

26. A pharmaceutical composition comprising a polypeptide of claim 2 for use in treating a glycogen storage disease.

27-30. (canceled)


SEQUENCE LISTING (4 sequences)

SEQ ID NO: 1 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 2 - protein, 18 aa, Homo sapiens
QQGASRPGPRDAQAHPGR

SEQ ID NO: 3 - protein, 16 aa, Homo sapiens
VPTQCDVPPNSRFDCA

SEQ ID NO: 4 - protein, 18 aa, Homo sapiens
IKDPANRRYEVPLETPRV